(19) United States
(12) Patent Application Publication
(10) Pub. No.: US 2025/0248369 A1
(43) Pub. Date: Aug. 7, 2025

(54) DEVICE FOR DETECTING WAIST AND BACK PAINS IN SMALL ANIMALS

(71) Applicant: SHENZHEN INSTITUTES OF ADVANCED TECHNOLOGY CHINESE ACADEMY OF SCIENCES, SHENZHEN (CN)

(72) Inventors: TIANWEN HUANG, SHENZHEN (CN); JIN KE, SHENZHEN (CN); SUNWOOK MOON, SHENZHEN (CN)

(21) Appl. No.: 19/189,199

(22) Filed: Apr. 24, 2025

Related U.S. Application Data
(63) Continuation of application No. PCT/CN2022/128528, filed on Oct. 31, 2022.

(51) Int. Cl. A01K 29/00; A61B 5/00

(52) U.S. Cl. CPC A01K 29/005; A61B 5/4824

(57) ABSTRACT

The present disclosure relates to a device for detecting waist and back pains in small animals, including a plurality of detection boxes. Each of the detection boxes includes a box body and a flip cover reversibly disposed on a top of the box body. The box body defines an accommodating groove for accommodating a small animal. The flip cover is a hollow structure. The device includes plurality of independent detection boxes, which allows for simultaneous detection of the waist and back pains on a plurality in small animals, thus improving a detection efficiency of the experimenter. The device transfers a conventional stimulation site from soles of feet to the waist and back, and is closer to a pathogenesis of chronic waist and back pains, and improves a detection accuracy of chronic waist and back pains.

### CROSS-REFERENCE TO RELATED APPLICATIONS

This application is a continuation of co-pending International Patent Application Number PCT/CN2022/128528, filed on Oct. 31, 2022, the disclosure of which is incorporated herein by reference in its entirety.

### TECHNICAL FIELD

This application relates to technical fields of animal experiment equipments, and in particular to a device for detecting waist and back pains in small animals.

### BACKGROUND

Waist and back pains are a common form of chronic pain caused by intervertebral disc degeneration (e.g., lumbar disc herniation) and soft tissue injuries of the waist. Early studies were mainly based on large animals (e.g., cats, pigs, and dogs), but they were costly and the experimental period was long. In recent years, mice and rats have been increasingly used as animal models for waist and back pains, but there is still a lack of effective methods for behavioural assessment. Currently, the main behavioural paradigms for detecting waist and back pains include von frey (mechanical prick test) and gait analysis. However, the traditional von frey detection mainly stimulates the soles of the feet of mice and rats, and although the reflex threshold of the soles of the feet can respond to a part of waist and back pains, it is difficult to directly reflect the true pain level of the waist and back pains, as the stimulation site is far away from the waist and back injury site. In other words, there is a lack of a back behavioural device that can directly detect waist and back pains, and the mechanical threshold of chronic waist and back pains in small animals cannot be detected directly.

### SUMMARY

One purpose of the present disclosure is to provide a device for detecting waist and back pains in small animals, which can directly, efficiently, and flexibly detect the mechanical threshold of chronic waist and back pains in small animals, which is conducive to further refine the behavioural paradigm of chronic waist and back pains, and to improve the accuracy and efficiency of the detection of chronic waist and back pains.

The present disclosure provides a device for detecting waist and back pains in small animals including a plurality of detection boxes. Each detection box includes a box body and a flip cover reversibly disposed on a top of the box body. The box body defines an accommodating groove formed for accommodating a small animal. The flip cover is a hollow structure to facilitate stimulation experiments on a waist and back of the small animal placed within the accommodating groove.

In at least one embodiment of the present disclosure, the device for detecting waist and back pains in small animals includes 8 to 10 of the detection boxes.

In at least one embodiment of the present disclosure, the hollow structure of the flip cover is a wire mesh structure.

In at least one embodiment of the present disclosure, the wire mesh structure has a wire diameter of 0.8 mm and a mesh aperture of 8*8 mm.

In at least one embodiment of the present disclosure, the box body of the detection box is a quadrangular structure, a length, width and height of the box body is 6*5.5*4 cm, and a thickness of the box body is 2 mm; and a length and a width of the wire mesh structure is 6*5.5 cm.

In at least one embodiment of the present disclosure, the box body includes five opaque acrylic plates.

In at least one embodiment of the present disclosure, one side of the flip cover is rotatably coupled to the box body, and the other side of the flip cover forms a removable connection with the box body by using any one of structures of magnetic members, snaps, Velcro tapes, and screws.

The device for detecting waist and back pains in small animals is provided with a reversible flip cover at a top of the detection box, which can facilitate a placement of a small animal, and a position of the flip cover corresponds to a position of a back of the small animal, so that it is convenient for an experimenter to directly use a series of von frey needles of different intensities to stimulate a waist and back of the small animal, and to observe a twisting of the small animal or a contraction response of the back of the small animal to assess the mechanical threshold of the small animal for chronic waist and back pains. Moreover, a plurality of detection boxes can satisfy a demand for conducting detection experiments of waist and back pains on a plurality of small animals at the same time, and improve a detection efficiency of the experimenters. The present disclosure provides the device that can directly, efficiently, and flexibly detect the mechanical threshold of chronic waist and back pains, which is conducive to further refine the behavioral paradigm of chronic waist and back pains, and improve the accuracy and efficiency of detection of chronic waist and back pains.

The purpose and advantages of the present disclosure will be further clarified through understanding the following description and accompanying drawings.

### BRIEF DESCRIPTION OF DRAWINGS

FIG. 1 illustrates a schematic structural view of a detection box of a device for detecting waist and back pains in small animals of a preferred embodiment of the present disclosure.

FIG. 2 illustrates a schematic view of a use of the device for detecting waist and back pains in small animals shown in FIG. 1 for performing experiments on waist and back pains in a small animal.

The description of reference signs: detection box 10; box body 11; opaque acrylic plate 111; flip cover 12; circular magnetic button 20; von frey filament 30; mice back site 40.

### DESCRIPTION OF EMBODIMENTS

The following description is used to expose the present disclosure so that a person skilled in the art can realize the application. The preferred embodiments in the following description are intended as examples only, and other obvious variations can be thought of by those skilled in the art. The basic principles of the present disclosure defined in the following description may be applied to other implementation solutions, transformation solutions, improvement solutions, equivalence solutions and other technical solutions that do not depart from the spirit and scope of the present disclosure.

It should be understood by those skilled in the art that, in the present disclosure, the terms ‘vertical orientation’, ‘orthogonal orientation’, ‘up’, ‘down’, ‘front’, ‘back’, ‘left’, ‘right’, ‘vertical’, “horizontal”, “top”, “bottom” ‘inside’, ‘outside’ and the like indicate orientations or positional relationships based on those shown in the accompanying drawings, which are only for the purpose of facilitating the description of the present disclosure and simplifying the description, and are not indicative of or suggestive of the necessity for the device or element referred to to be of a particular orientation, to be constructed and operated in a particular orientation, and therefore the above terms are not to be construed as a limitation of the present disclosure.

It is to be understood that the term ‘one’ is to be understood as ‘at least one’ or ‘one or more’, i.e., in one embodiment, the number of one element may be one, and in another embodiment, the number of the element may be more than one, and the term ‘one’ is not to be understood as a limitation on the number.

In the description of the present disclosure, it is to be noted that, unless otherwise expressly specified and limited, the terms ‘mounted’, ‘coupled with each other’, ‘coupled’ are to be understood in a broad sense, e.g., as a fixed connection or as a detachable connection, or an integral connection; it may be a mechanical connection, an electrical connection or a connection that can communicate with each other; it may be a direct connection or an indirect connection through an intermediate medium; it may be a connection within the two elements or an interaction between the two elements. For those of ordinary skill in the art, the specific meanings of the above terms in the present disclosure may be understood on a case-by-case basis.

The traditional detection device for detecting mechanical pain threshold mainly detects a mechanical pain threshold by using sole stimulation, and thus its hollow structure is set at a bottom of a detection cage, and because the sole stimulation site is far away from the waist and back injury site, it is difficult to accurately assess the true pain level of waist and back pains in terms of anatomical position. The present disclosure therefore provides a detection device for waist and back behavioral, in particular a portable detection device capable of directly detecting the mechanical threshold of chronic waist and back pains in small animals, in order to facilitate the efficient and flexible detection of the mechanical threshold of chronic waist and back pains in small animals, so as to further refine the behavioral paradigm of chronic waist and back pains, and to improve the accuracy and efficiency of the detection of chronic waist and back pains.

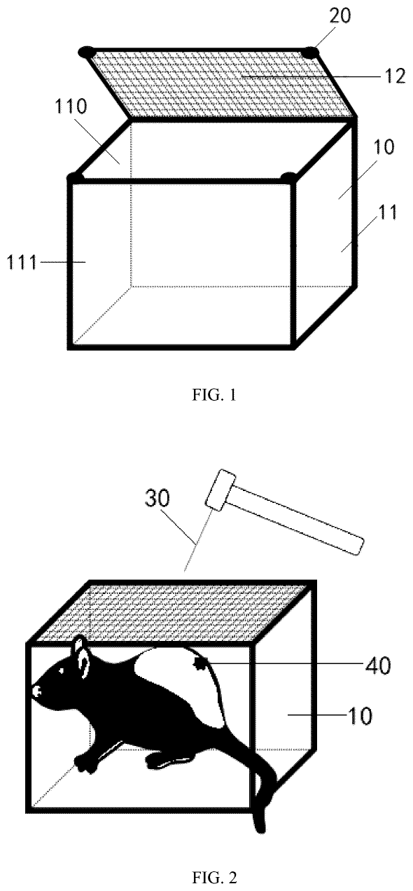

As shown in FIGS. 1 and 2, the specific structure and use of the device for detecting waist and back pains in small animals according to a preferred embodiment of the present disclosure is specifically elucidated.

It is to be understood that the device of the present disclosure is suitable for detecting waist and back pains in small animals such as mice and rats, rabbits, etc., and the present disclosure does not limit the types of experimental animals for which the device can be used. The following is a detailed description of the specific structure and the using method of the device described taking the device for detecting waist and back pains in small animals for use in the detection of waist and back pains in mice as an example.

Specifically, the device for detecting waist and back pains in small animals includes a plurality of detection boxes 10. The structure of a single detection box 10 is shown in FIG. 1. Each detection box 10 includes a box body 11 and a flip cover 12 reversibly disposed on a top of the box body 11. The box body 11 defines an accommodating groove 110 for accommodating a small animal. The flip cover 12 defines a hollowed structure to facilitate stimulation experiments, such as filament or needle puncture experiments, on the waist and back of the small animal placed within the accommodating groove 110.

In at lease one embodiment of the present disclosure, the device for detecting waist and back pains in small animals includes 8 to 10 of the detection boxes 10, and each detection box 10 can be numbered to facilitate experimental recording and observation by an experimenter. The quantity of the detection boxes 10 may be added or reduced according to the specific needs of the experiment, and the size of the detection boxes 10 may also be set according to the needs of the actual experiment, and the present disclosure does not impose any limitation on either the quantity or the size of the detection.

Specifically, in this specific embodiment, the box body 11 of the detection box 10 is a quadrangular structure, i.e., the detection box 10 is a small square box, and the box body 11 is specifically composed of five opaque acrylic plates 111, with a length, width, and height of 6*5.5*4 cm, and a thickness of a single opaque acrylic plate 111 is 2 mm, i.e. a thickness of the box body 11 is 2 mm.

Correspondingly, a length and width of the flip cover 12 is 6*5.5 cm, and in this specific embodiment, the hollowed structure of the flip cover 12 is a wire mesh structure, and the wire mesh structure has a wire diameter of 0.8 mm and a mesh aperture of 8*8 mm. It should be understood that the wire diameter and mesh aperture of the wire mesh structure can be set according to actual experimental requirements, without limited herein.

In particular, one side of the flip cover 12 is rotatably coupled to the box body 11, and the other side of the flip cover 12 is detachably coupled to the box body 11 by using any one of magnetic members, snaps, Velcro, and screws.

In this specific embodiment, the other side of the flip cover 12 and the box body 11 are magnetically coupled in a detachable manner, specifically, two corner points of the other side of the flip cover 12 and two opposite corner points of the box body 11 are respectively provided with circular magnetic buttons 20, and the circular magnetic buttons 20 of the flip cover 12 and the circular magnetic buttons 20 of the box body 11 are magnetically opposite, which on the one hand facilitates the experimenter to open the flip cover 12 and conveniently put the mice in the accommodating groove 110, and on the other hand enables the flip cover 12 to form a stable connection with the box body 11 through the mutual attraction of the circular magnetic buttons 20 when the flip cover 12 closes on the box body 11, preventing the mice inside the box body 11 from opening the flip cover 12.

In some embodiments of the present disclosure, either of the flip cover 12 and the box body 11 is provided with a magnetic member, and the other of the flip cover 12 and the box body 11 is provided with a material capable of being attracted by the magnetic member, such as a metal, etc., at a corresponding position, so that magnetic connection between the flip cover 12 and the box body 11 can also be realized. The magnetic member may be triangular, quadrilateral, pentagonal or irregularly shaped, without limited herein.

As shown in FIG. 2, the detail using process of the device for detecting waist and back pains in small animals is as follows:

The flip cover 12 is opened and the mice is placed in the accommodating groove of the box body 11, and the flip cover 12 is covered on the accommodating groove of the box body 11 well;

The mice is in an arched back state in the detection box 10 due to instinctive behavior;

The mice is stimulated on its mice back site 40 (the site of the back L4-6) using von frey filaments 30 with different intensities, and a twisting of the mice or a contraction response of the back of the mice is observed to assess the mechanical threshold of chronic waist and back pains in the mice.

It is to be noted that the magnitude of the mechanical threshold for chronic waist and back pains in mice can be assessed based on a strength of the contraction of the back of the mice.

It is to be understood that since the device for detecting waist and back pains in small animals of the present disclosure includes a plurality of independently detection boxes 10, it is possible to carry out experiments for detecting waist and back pains on a plurality of small animals at the same time to improve the efficiency of detection by the experimenter; and the device for detecting waist and back pains in small animals transfers the traditional stimulation site from the soles of the feet to the waist and back, which is closer to the pathogenesis of chronic waist and back pains, and improves the accuracy of the detection of the chronic waist and back pains.

In general, the present disclosure provides an animal waist and back behavioral detection device, specifically a device for detecting waist and back pains in small animals, capable of efficiently and flexibly detecting the mechanical threshold of chronic waist and back pains in small animals, which is conducive to further refine the behavioral paradigm of chronic waist and back pains, and improve the accuracy and efficiency of the detection of the chronic waist and back pains.

The various technical features of the above embodiments can be combined in any way, and all possible combinations of the various technical features of the above embodiments have not been described in order to make the description concise; however, as long as there is no contradiction in the combinations of these technical features, they should be considered to be within the scope of the present specification.

The above embodiments only express the preferred embodiments of the present disclosure, which are described in a more specific and detailed manner, but are not to be construed as a limitation of the scope of the present disclosure. It should be pointed out that, for a person of ordinary skill in the art, a number of deformations and improvements can be made without departing from the conception of the present disclosure, all of which fall within the protection scope of the present disclosure. Therefore, the protection scope of the present disclosure shall be subject to the attached claims.

What is claimed is:

1. A device for detecting waist and back pains in small animals, wherein comprises:
a plurality of detection boxes, each detection box comprising a box body and a flip cover reversibly disposed on a top of the box body;
wherein the box body defines an accommodating groove formed configured for accommodating a small animal;
wherein the flip cover is a hollow structure.

2. The device for detecting waist and back pains in small animals according to claim 1, wherein the device for detecting waist and back pains in small animals comprises 8 to 10 of the detection boxes.

3. The device for detecting waist and back pains in small animals according to claim 1, wherein the hollow structure of the flip cover is a wire mesh structure.

4. The device for detecting waist and back pains in small animals according to claim 3, wherein the wire mesh structure has a wire diameter of 0.8 mm and a mesh aperture of 8*8 mm.

5. The device for detecting waist and back pains in small animals according to claim 4, wherein the box body of the detection box is a quadrangular structure, a length, width and height of the box body is 6*5.5*4 cm, and a thickness of the box body is 2 mm; and a length and width of the wire mesh structure is 6*5.5 cm.

6. The device for detecting waist and back pains in small animals according to claim 5, wherein the box body comprises five opaque acrylic plates.

7. The device for detecting waist and back pains in small animals according to claim 1, wherein one side of the flip cover is rotatably coupled to the box body, and the other side of the flip cover forms a removable connection with the box body by using any one of structures of magnetic members, snaps, Velcro tapes, and screws.

8. The device for detecting waist and back pains in small animals according to claim 2, wherein one side of the flip cover is rotatably coupled to the box body, and the other side of the flip cover forms a removable connection with the box body by using any one of structures of magnetic members, snaps, Velcro tapes, and screws.

9. The device for detecting waist and back pains in small animals according to claim 3, wherein one side of the flip cover is rotatably coupled to the box body, and the other side of the flip cover forms a removable connection with the box body by using any one of structures of magnetic members, snaps, Velcro tapes, and screws.

10. The device for detecting waist and back pains in small animals according to claim 4, wherein one side of the flip cover is rotatably coupled to the box body, and the other side of the flip cover forms a removable connection with the box body by using any one of structures of magnetic members, snaps, Velcro tapes, and screws.

11. The device for detecting waist and back pains in small animals according to claim 5, wherein one side of the flip cover is rotatably coupled to the box body, and the other side of the flip cover forms a removable connection with the box body by using any one of structures of magnetic members, snaps, Velcro tapes, and screws.

12. The device for detecting waist and back pains in small animals according to claim 6, wherein one side of the flip cover is rotatably coupled to the box body, and the other side of the flip cover forms a removable connection with the box body by using any one of structures of magnetic members, snaps, Velcro tapes, and screws.
